(19) United States
(12) Patent Application Publication
(10) Pub. No.: US 2011/0028754 A1
(43) Pub. Date: Feb. 3, 2011

(54) PROCESS FOR HETEROGENEOUSLY CATALYZED ESTERIFICATION OF (METH)ACRYLIC ACID WITH OXYALKYLATED POLYOLS

(75) Inventors: Michael Ludewig, Leverkusen (DE); Jan Weikard, Ordenthal-Erberich (DE); Andre Düx, Bruhl (DE); Benie Marotz, Dusseldorf (DE); Georg Ronge, Dusseldorf (DE); Aurel Wolf, Wulfrath (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/671,989

(22) PCT Filed: Jul. 29, 2008
(86) PCT No.: PCT/EP08/06224

§ 371 Date: Feb. 3, 2010

(30) Foreign Application Priority Data
Aug. 7, 2007 (DE) 10 2007 037 140.5

(51) Int. Cl. C07C 69/54

(52) U.S. Cl. 560/224

(57) ABSTRACT

The present invention relates to a process for heterogeneously catalyzed partial esterification of (meth)acrylic acid with oxyalkylated polyols, wherein the oxyalkylated polyols have at least 3 free hydroxyl groups, and wherein the catalyst is selected from the group comprising acidic ion exchange resins and/or acidic zeolites. The invention further relates to (meth)acrylic esters and urethane acrylates, obtained by a process according to the present invention, and to the use thereof as radiation-curable compounds.

The present invention relates to a process for heterogeneously catalysed esterification of (meth)acrylic acid with oxyalkylated polyols, said oxyalkylated polyols having at least three free hydroxyl groups. It further relates to (meth)acrylic esters obtained by a process according to the present invention and to the use of (meth)acrylic esters according to the present invention as radiation-curable compounds.

Radiation-curable coating compositions based on reaction products of hydroxy-functional esters of (meth)acrylic acid and isocyanates are referred to as urethane acrylates. Of particular interest for use as coating compositions are esters of (meth)acrylic acid with oxyalkylated polyols.

The synthesis of hydroxy-functional partial esters of (meth)acrylic acid proceeding from a mixture of differently oxyalkylated polyols is described, for example, in EP 0 900 778 A1. This document relates to a process for preparing esters of mono- or polyhydric alcohols or ester precursors having at least two hydroxyl groups per molecule, prepared from polyhydric alcohols and mono- or dibasic, saturated or aromatically unsaturated carboxylic acids and ethylenically unsaturated carboxylic acids such as (meth)acrylic acid by acid-catalysed azeotrope esterification, the catalyst acid and carboxylic acids unconverted during the esterification being converted by subsequent reaction with ethylenically unsaturated monoepoxides. The acidic esterification catalysts used are inorganic or organic acids. Examples thereof are sulphuric acid, phosphoric acid, pyrophosphoric acid, p-toluenesulphonic acid, styrenedivinylbenzenesulphonic acid, chlorosulphonic acid and chloroformic acid.

However, the use of an acid catalyst in homogeneous phase brings disadvantages. For instance, the catalyst has to be removed after the reaction, since the end product would otherwise be contaminated. A particular problem is the reaction of the remaining acid with isocyanates in the synthesis of the urethane acrylate. The excess acid can be removed, for example, by extractive washing with water, by reaction with epoxides or by reaction with carbodiimides. However, what is common to all of these processes is that they require an additional process step.

The use of a heterogeneous catalyst is described in DE 103 17 435 A1. This document relates to a process for preparing (meth)acrylic esters by heterogeneously catalysed reaction of (meth)acrylic acid with at least one alcohol in a reactor, in which the water content in the bottoms of the azeotrope column is less than 0.15 ppm by weight and/or the content of (meth)acrylic acid in the bottoms of the azeotrope column is not more than 60% by weight and/or the heterogeneous catalyst is contacted before the reaction with a stabilizer-containing alcohol solution. Useful heterogeneous catalysts include all strongly acidic ion exchange resins and all acidic zeolites. Preferred ion exchange resins are styrene-divinylbenzene polymer resins with sulphonic acid groups. Preferred acidic zeolites are those which comprise a crystalline metal silicate in protonated form, for example aluminium silicate, boron silicate, iron silicate, gallium silicate in the H form. According to this document, the alcohol used may in principle be any alcohol containing 1 to 12 carbon atoms.

The (meth)acrylic esters obtained in this way can be purified by distillation. In general, they form minimum azeotropes with the water of reaction obtained by the esterification and can be removed as top products. It is not taught how (meth)acrylic esters with higher molecular weight which cannot be distilled off can be obtained by means of heterogeneous catalysis. However, esters of interest include those of (meth)acrylic acid with oxyalkylated polyols, which can serve as valuable intermediates in the synthesis of the corresponding urethane acrylate. Of particular interest are those esters with oxyalkylated polyols which have at least three free hydroxyl groups and wherein an average of one hydroxyl group is still present in free form after the esterification reaction. Such a controlled heterogeneously catalysed partial esterification has not yet succeeded satisfactorily to date. This should also be considered against the background that oxyalkylated polyols are not suitable for conversion by every catalyst owing to their polarity and their ability to form coordinate bonds.

Accordingly, the present invention has for its object to provide a process for heterogeneously catalysed partial esterification of (meth)acrylic acid with oxyalkylated polyols which have at least three free hydroxyl groups, wherein no acid traces for removal are present in the reaction product and wherein the reaction product can therefore be subjected directly to a urethanization of a remaining free hydroxyl group.

The object is achieved in accordance with the invention by a process for heterogeneously catalysed partial esterification of (meth)acrylic acid with oxyalkylated polyols, said oxyalkylated polyols having at least three free hydroxyl groups and said catalyst being selected from the group comprising acidic ion exchange resins and/or acidic zeolites.

Oxyalkylated polyols in the context of the present invention may be based on trihydric or higher polyhydric alcohols. Preferred base molecules used are glycerol, trimethylolpropane (TMP), pentaerythritol, ditrimethylolpropane, dipentaerythritol and/or sorbitol. The oxyalkylation can proceed by known methods of synthesizing polyethers. Preferred monomers here are ethylene oxide, propylene oxide and/or tetrahydrofuran, although different monomers can also be used in succession in order to obtain blocks.

The at least 3 free hydroxyl groups of the oxyalkylated polyols should be understood as being specified per molecule. They may reside in the terminal position on the polyether chain or else form a branch. It is possible, for example, for 3, 4, 5 or 6 free hydroxyl groups to be present per molecule. The oxyalkylated polyols may also be present as a mixture of different compounds. In that case, the statement that at least 3 free hydroxyl groups are present means a statistical average.

In a partial esterification, not all free hydroxyl groups are esterified. For instance, an average of ≧2 to ≦2, preferably ≧0.5 to ≦1.5 and more preferably ≧0.8 to ≦1.2 free hydroxyl groups may remain in the ester.

The inventive catalyst is selected from the group comprising acidic ion exchange resins and/or acidic zeolites. It is thus a heterogeneous catalyst.

Suitable ion exchange resins are prepared from a polymerizable vinyl component and a crosslinker. The vinyl components used may be styrene, a-methylstyrene, vinyltoluene, ethylvinylbenzene or similar compounds. Crosslinkers possess at least two polymerizable groups and may be selected, for example, from the group comprising divinylbenzene, divinyltoluene, trivinylbenzene, divinylchlorobenzene, divinylxylene and/or divinylnaphthalene. Preference is given to preparing ion exchange resins from styrene and divinylbenzene, in which case the proportion of the crosslinker may be ≧0.1% by weight to ≦20% by weight, based on the overall monomer mixture, and preferably ≧1 to ≦10% by weight. The polymer resins contain sulphonic acid and/or carboxyl groups, sulphonic acid groups being particularly suitable. The concentration of sulphonic acid groups may be ≧0.1 mol/l to ≦3 mol/l and preferably ≧0.2 mol/l to ≦2 mol/l of resin. The particle size of the acidic ion exchangers may be ≧100 μm to ≦5000 μm and preferably ≧200 μm to ≦2500 μm.

Suitable acidic zeolites are crystalline metal silicates which are preferably present in protonated form. The generally trivalent metal from the metal silicalite may, without being restricted thereto, be selected from the group of Al, B, Fe, Ga. Metal silicates may, however, also comprise two or more of these metals. Typically, it is possible to use Y, beta, ZSM-5 and/or mordenite zeolites, which are preferably present in the H form.

The catalyst may be present in free or immobilized form in the reaction mixture or be immobilized in additional external apparatus such as distillation columns or fixed bed reactors.

By means of the catalyst selected in accordance with the invention, it becomes possible to perform the desired partial esterification of the oxyalkylated polyols and to obtain the desired reactants for a subsequent urethanization, without troublesome residues of a homogeneous acid catalyst being present. Consequently, a purification step is saved.

The process according to the invention may be configured as a continuous process or as a batchwise process.

It is favourable to introduce an oxygenous gas, preferably air or mixtures of oxygen and inert gases, into the solvent-containing reaction mixture during the performance of the process according to the invention. The addition of oxygen activates inhibitor present in the reaction mixture, which is intended to prevent the polymerization of the (meth)acrylic acid and/or esters thereof.

The process according to the invention is carried out in a solvent which is immiscible with water and is distillable with water for the purposes of a steam distillation. This solvent is also referred to as azeotroping agent. It can form azeotropic mixtures with water. For this purpose, hydrocarbons and the halogen or nitro substitution products thereof are useful, as are further solvents which neither react with the reactants nor are modified under the influence of the acidic catalyst.

Advantageously, unsubstituted hydrocarbons are used. Examples include: aliphatic hydrocarbons such as hexane, heptane, octane, isooctane, petroleum fractions of various boiling ranges, cycloaliphatic hydrocarbons such as cyclopentane, cyclohexane and/or methylcyclohexane, or aromatic hydrocarbons such as benzene, toluene and/or the isomeric xylenes. Preferably, those solvents which boil within the range from ≧70° C. to ≦120° C. are used. Mention should be made here in particular of isooctane, cyclohexane, toluene or petroleum fractions in the boiling range of ≧70° C. to ≦120° C. The water-immiscible solvent may also be a mixture of the abovementioned substances. An amount of ≧5% by weight to ≦100% by weight, preferably ≧10% by weight to ≦80% by weight, more preferably ≧20% by weight to ≦60%, based on the weight of the reaction components to be esterified, is used.

To stabilize the (meth)acrylic acid and esters thereof against unwanted polymerization, it is favourable to perform the process according to the invention in the presence of one or more polymerization inhibitors. The inhibitor, also referred to as a stabilizer, can be added in an amount of ≧0.01% by weight to ≦5% by weight, preferably of ≧0.05% by weight to ≦2% by weight, more preferably of ≧0.1% by weight to ≦1.5% by weight, based on the mixture of (meth)\acrylic acid and polyol to be esterified.

Suitable stabilizers may be selected from the group comprising sodium dithionite, sodium hydrogensulphide, sulphur, hydrazine, phenylhydrazine, hydrazobenzene, N-phenyl-α-naphthylamine, N-phenylethanolamine, dinitrobenzene, picric acid, p-nitrosodimethylaniline, diphenylnitrosamine, phenols, p-tert-butylparacatechol, 2,5-di-tert-amylhydroquinone, p-alkoxyphenols, 4-methoxyphenol, di-tert-butylhydroquinone, tetramethylthiuram disulphide, 2-mercaptobenzothiazole, phenothiazine, hydroquinone monomethyl ether and/or dimethyldithiocarbamic acid sodium salt.

In an advantageous embodiment of the present invention, the oxyalkylated polyols have a degree of oxyalkylation of ≧1 to ≦30, preferably of ≧3 to ≦25. The degree of oxyalkylation refers to the amount of oxyalkylation monomer based on the amount of alcohol. For example, 7 mol of ethylene oxide per mole of trimethylolpropane would correspond to a degree of oxyalkylation of 7. Such oxyalkylated polyols may have a molecular weight of, for example, ≧600 g/mol to ≦5000 g/mol. They have the property that their partial or full esters of (meth)acrylic acid cannot be distilled under the customary process conditions. They are therefore particularly suitable for use in the process according to the invention.

In a further advantageous embodiment of the process according to the invention, the catalyst further comprises a stabilizer which is capable at least of slowing the polymerization of (meth)acrylic acid. Moreover, the concentration of the stabilizer on and/or in the catalyst is higher than the concentration of the stabilizer in the reaction solution. This can prevent (meth)acrylic acid from being polymerized on the catalyst and hence inactivating it. For example, before the catalyst is first contacted with the remaining reactants, a solution of the stabilizer in the oxyalkylated polyol used can be conducted over the catalyst until the catalyst is saturated with the stabilizer. Suitable stabilizers have already been mentioned above.

The concentration of the stabilizer in the oxyalkylated polyol may, during the contacting, be within a range of ≧0.01% by weight to ≦5% by weight, preferably of ≧0.05% by weight to ≦2% by weight, more preferably of ≧1% by weight to ≦1.5% by weight.

In a further advantageous embodiment of the present invention, the reaction conditions are selected from the group comprising:
the pressure is ≧0.5 bar to ≦5 bar, preferably ≧0.9 bar to ≦2 bar;
the temperature is ≧50° C. to ≦150° C., preferably ≧80° C. to ≦120° C.; and/or
the molar ratio of (meth)acrylic acid to OH groups is ≧1:3 to ≦3:3, preferably ≧1.5:3 to ≦2.5:3

In a further advantageous embodiment of the present invention, the catalyst is immobilized in a distillation column. The immobilization in the distillation column can be carried out, inter alia, by means of reactive structured packings, random packings or distillation trays with specific devices. Examples of reactive structured packings, random packings or column trays can be found in Ullmann, Encyclopedia of Industrial Chemistry, Seventh Release, 2007. The reaction mixture is introduced at the upper end of the distillation column. The amount of mixture introduced per hour may be in the order of magnitude of ≧0.1 times to ≦50 times, preferably of ≧0.5 times to ≦20 times and even more preferably of ≧1 times to ≦10 times the volume of the reactor. The advantage of this procedure, which is also referred to as reactive distillation, lies in a simplification of the apparatus construction.

In a further advantageous embodiment of the present invention, the catalyst is suspended in the reaction medium. What is advantageous about this is especially the good distribution of the catalyst in the reaction chamber.

In a further advantageous embodiment of the process according to the invention, the catalyst is immobilized in the reaction medium. The catalyst can, for example, be accommodated in pockets produced from wire mesh. These pockets can then be mounted in the reactor on the stirrer, on the baffles and/or as separate internals. What is advantageous about this is especially that the catalyst can be removed easily, in order to be exchanged or regenerated. In the case of the ion exchange resins, the regeneration is effected by elution with a strong acid, for example sulphuric acid, hydrochloric acid and/or nitric acid. Preference is given to using sulphuric acid, it being possible to use either concentrated or dilute acid. Zeolites, in contrast, are regenerated by calcinations in an oxygenous atmosphere at temperatures of ≧300° C., preferably of ≧400° C. to ≦500° C.

In a further advantageous embodiment of the present invention, the esterification is followed by the reaction of the resulting (meth)acrylic ester with an isocyanate, preferably with isophorone diisocyanate, hexamethylene 1,6-diisocyanate (HDI), diphenylmethane 4,4′-diisocyanate tolylene 2,4-diisocyanate (TDI) and/or tolylene 2,6-diisocyanate (TDI). The commercially desired urethane acrylates are thus synthesized. The advantage of the process according to the invention is shown here, by virtue of which, after the easily accomplished removal of the heterogeneous catalyst, no acid traces for removal enter into troublesome side reactions with the isocyanates.

The present invention further provides (meth)acrylic esters obtained by a process according to the present invention. Accordingly, the present invention also relates to urethane acrylates which are obtained by a process according to the present invention.

The invention further provides for the use of (meth)acrylic esters according to the present invention as radiation-curable compounds, preferably as binders for coating surfaces and/or articles. Accordingly, the present invention also relates to the use of urethane acrylates according to the present invention as radiation-curable compounds, preferably as binders for coating surfaces and/or articles. In this connection, radiation-curable relates, inter alia, to curing by means of heat, visible light, UV rays and/or electron beams.

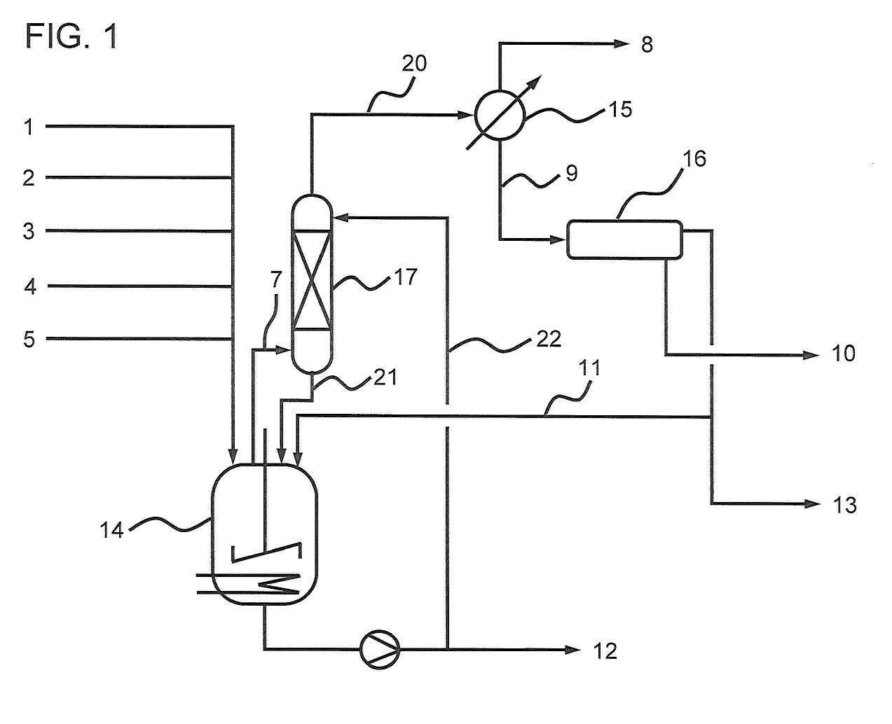

The present invention is illustrated further hereinafter with reference to FIGS. 1 to 3.

The figures show:

FIG. 1 a process according to the invention wherein the catalyst is immobilized in a distillation column

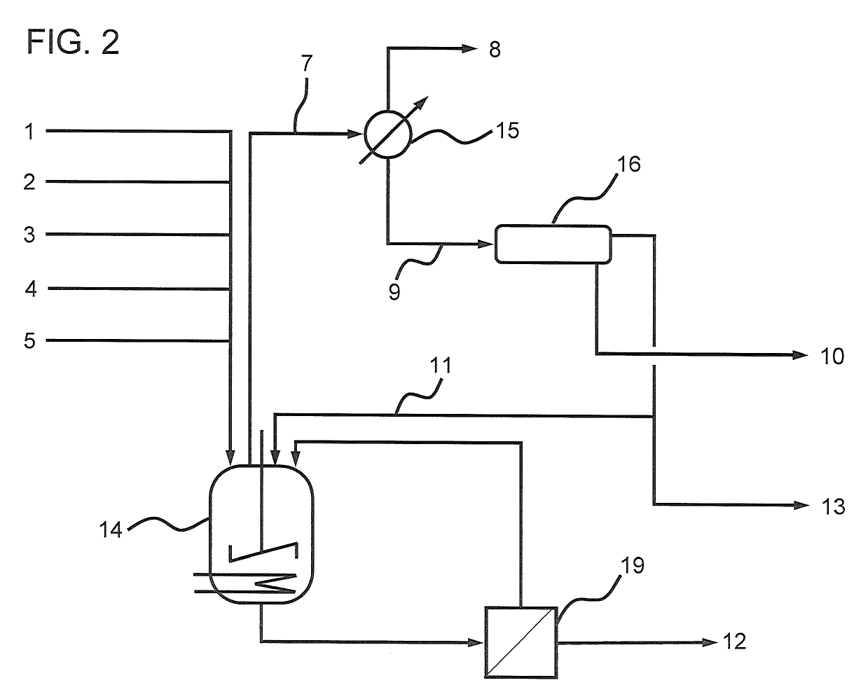

FIG. 2 a process according to the invention wherein the catalyst is suspended in the reactor

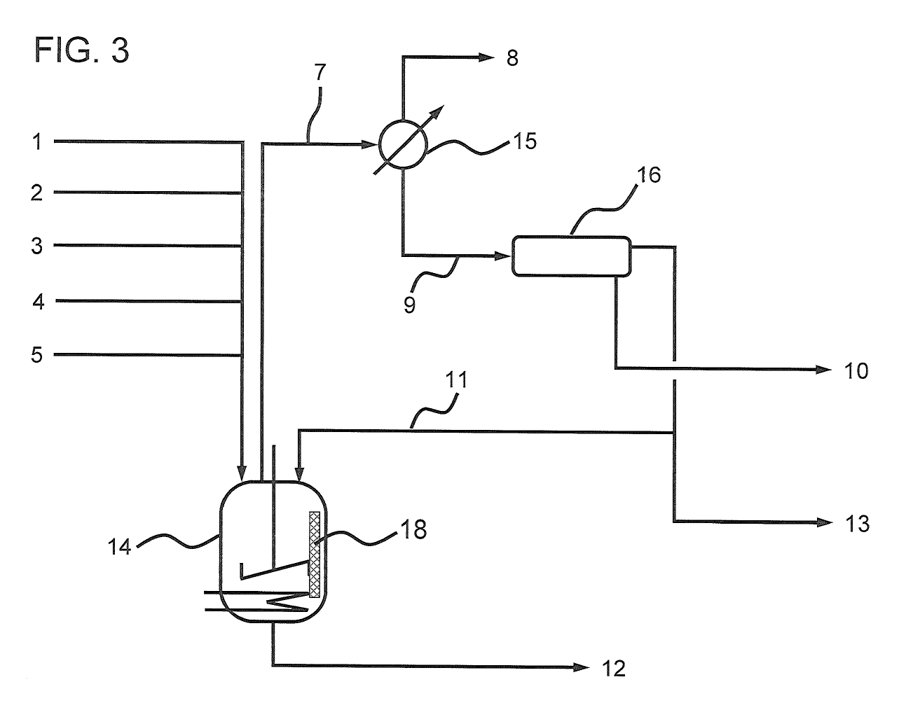

FIG. 3 a process according to the invention wherein the catalyst is immobilized in the reactor

In the figures, the following reference numerals are used:
1 oxyalkylated polyols
2 (meth)acrylic acid
3 azeotroping agent
4 polymerization inhibitor
5 oxygenous gas
6 (not used)
7 vapour mixture from reactor
8 gaseous constituents
9 distillate
10 water of reaction
11 return line of the azeotroping agent into the reactor
12 product
13 removal of the azeotroping agent from the process
14 reactor
15 condenser
16 liquid/liquid separation
17 distillation column with immobilized heterogeneous catalyst
18 heterogeneous catalyst
19 filter apparatus
20 vapour mixture
21 effluent of reaction mixture from distillation column
22 stream

FIG. 1 shows a process according to the invention wherein the heterogeneous catalyst is immobilized in a distillation column. The reactor (14) has apparatus for heating and for stirring. The reactants are metered into the reactor (14) in the following sequence: oxyalkylated polyols (1), polymerization inhibitor (4), azeotroping agent (3) and (meth)acrylic acid (2). In addition, oxygenous gas (5) is passed through the reactor (14). The heterogeneous catalyst is immobilized in a tray column or in a column with structured or random packing. The heterogeneous catalyst can be contacted additionally with polymerization inhibitor before or immediately after the first filling. After the metered addition, the mixture present in the reactor (14) is stirred and introduced at the upper end of the distillation column (17) which in this case also comprises the catalyst. This is shown by stream (22). The amount of the mixture introduced per hour may correspond to ≧0.1 to ≦50 times, preferably ≧0.5 to ≦20 times and more preferably ≧1 times to ≦10 times the volume of the reactor. Stream (21) is the effluxing reactant mixture. Since the catalyst is already immobilized, no additional equipment for removal is required.

This is followed by heating to boiling temperature. The water formed in the reaction is for the most part already removed from the liquid, reactive phase by the azeotroping agent (3) evaporated in the reactor (14). By virtue of the simultaneous reaction and water removal, the conversion in the reaction zone of the distillation column (17) is high. The water formed in the reaction leaves the top of the distillation column (17) as a vapour mixture (20) together with the azeotroping agent (3). In a condenser (15), the volatile constituents are condensed out. Remaining gaseous constituents (8) leave the condenser (15). The distillate (9) is subjected to a liquid/liquid separation (16). This separates the water of reaction (10) from the azeotroping agent. The azeotroping agent can either be withdrawn from the process (13) or be passed (11) back into the reactor (14) in substantially anhydrous form.

After the reaction has ended, the azeotroping agent is removed by distillation from the product (12). The azeotroping agent removed is condensed and sent to a suitable collecting vessel. To remove the product present in the holdup of the distillation column, it is operated under reflux. To this end, a portion of the condensed azeotroping agent is introduced at the top of the column.

FIG. 2 shows a process according to the invention wherein the heterogeneous catalyst is suspended in the reactor. The reactor (14) has apparatus for heating and for stirring. The reactor further comprises the heterogeneous catalyst in suspension. The sequence of metered addition of the reactants (1, 4, 3, 2) corresponds to the scheme from FIG. 1. In addition, oxygenous gas (5) is passed through the reactor (14). Before the first filling, the heterogeneous catalyst can be saturated with polymerization inhibitor. The heterogeneous catalyst is metered in in the manner known to those skilled in the art, for example by means of a star feeder, metering screw or manually.

After the metered addition, the mixture present in the reactor (14) is stirred and heated to boiling temperature, which starts the reaction. The water present in the reaction is removed from the reactor (14) by evaporation of an azeotroping agent (3) as already described in FIG. 1. The azeotroping agent can either be withdrawn (13) from the process or be passed (11) back into the reactor (14) in substantially anhydrous form. After the reaction has ended, the azeotroping agent is removed from the product by distillation. The azeotroping agent removed is condensed and sent to a suitable collecting vessel (13). The product (12) is removed from a heterogeneous catalyst mechanically by means of a filter device (19), for example by means of filtration or decantation. It is possible that a wash or regeneration of the catalyst removed follows.

FIG. 3 shows a process according to the invention wherein the heterogeneous catalyst is immobilized in the reactor. The reactor (14) has apparatus for heating and for stirring. The reactor further comprises the heterogeneous catalyst in suspension. The sequence of metered addition of the reactants (1, 4, 3, 2) corresponds to the scheme from FIG. 1. In addition, oxygenous gas (5) is passed through the reactor (14). The heterogeneous catalyst can be contacted with polymerization inhibitor before the first filling. The heterogeneous catalyst (18) is immobilized in vessels in the reactor. Suitable vessels are, for example, catalyst pockets produced from wire mesh. These may be mounted in the reactor on the stirrer and/or the baffles and/or as separate internals.

The water formed in the reaction is removed from the reactor (14) by evaporating an azeotroping agent (3) as already described in FIG. 1. The azeotroping agent can either be removed (13) from the process or be passed (11) back into the reactor (14) in substantially anhydrous form. After the reaction has ended, the azeotroping agent is removed from the product by distillation. The azeotroping agent removed is condensed and sent to a suitable collecting vessel (13). The product (12) is withdrawn directly from the reactor (14).

The present invention will be illustrated in detail with reference to the following working examples. The substance parameters reported are determined by the following test methods:

The acid number is reported in mg KOH/g of sample. It is determined by titration with 0.1 mol/l NaOH solution against bromothymol blue (ethanolic solution), colour change from yellow through green to blue, based on standard DIN 3682.

The hydroxyl number is reported in mg KOH/g of sample. It is determined by titration with 0.1 mol/l of methanolic KOH solution after low-temperature acetylation with acetic anhydride catalysed by dimethylaminopyridine based on standard DIN 53240.

The determination of the NCO content in % was undertaken on the basis of standard DIN EN ISO 11909 by back-titration with 0.1 mol/l hydrochloric acid after reaction with butylamine.

The viscosity measurements were carried out at 23° C. with a plate-plate rotary viscometer, RotoVisko 1 from Haake, Germany, to the standard ISO/DIS 3219:1990.

### EXAMPLE 1

### Heterogeneous Catalyst in Reactive Structure Packing

Into a heatable glass vessel of capacity 10 litres with stirrer, gas inlet and thermostat were weighed, while passing air through (1.0 times the apparatus volume per hour) and passing nitrogen over (2.0 times the apparatus volume per hour), 3975 g of an, on average, 12-tuply ethoxylated, trimethylpropane-started polyether (hydroxyl number 255; dynamic viscosity 265 mPa·s at 23° C.), 892.5 g of acrylic acid, 14.8 g of 4-methoxyphenol, 1 g of 2,5-di-tert-butylhydroquinone and 2557 g of isooctane. The reaction mixture was stirred and pumped to the top of a distillation column with a pumped circulation rate of 8 kg/h. This column had a diameter of 70 mm and was equipped in the upper segment with 2 m of reactive structured packing of the Katapak-SP11 type (manufacturer: Sulzer Chemtech Ltd), and in the lower segment with 2 m of structured packing of the Rombopak 6M type (manufacturer: Kühni AG). The catalyst pockets of the reactive packing were each charged with 73 g of Dowex 50W×4 20-50 catalyst, an acidic ion exchange resin. Heating to boiling temperature under standard pressure conditions (94° C.108° C.) started the reaction. Evaporation of isooctane (2500 g/h) removed the water formed in the reactive structured packing. After a run time of 21 h, the acid number had reached a value of <2.5. During this time, an amount of water of 223 g was separated out. Subsequently, the mixture was cooled to 50° C. The hydroxyl number of the product was 80.

### EXAMPLE 2

### Heterogeneous Catalyst Suspended in the Reaction Mixture

Into a heatable glass vessel of capacity 10 litres with stirrer, gas inlet and thermostat were weighed, while passing air through (1.0 times the apparatus volume per hour) and passing nitrogen over (2.0 times the apparatus volume per hour), 3975 g of an, on average, 12-tuply ethoxylated, trimethylpropane-started polyether (hydroxyl number 255; dynamic viscosity 265 mPa·s at 23° C.), 892.5 g of acrylic acid, 14.8 g of 4-methoxyphenol, 1 g of 2,5-di-tert-butylhydroquinone, 2557 g of isooctane and 200 g of Dowex 50W×2 100-200 heterogeneous catalyst, an acidic ion exchange resin. The reaction mixture was stirred heated to boiling temperature (94° C.-180° C.) under standard pressure conditions. Evaporation of isooctane (2500 g/h) removed the water formed in the reaction. After a run time of 19 h, the acid number had reached a value of <2.1. During this time, an amount of water of 220 g was separated out. Subsequently, the mixture was cooled to 50° C. The hydroxyl number of the product was 80.

### EXAMPLE 3

### Urethanization

A 1000 ml four-neck glass flask with reflux condenser, heatable oil bath, mechanical stirrer, air circulation (1 litre/h), internal thermometer and dropping funnel was initially charged with 545.78 g of the product from Example 2, 0.8 g of 2,6-di-tert-butyl-4-methylphenol, 86.68 g of hydroxyethyl acrylate and 0.8 g of dibutyltin dilaurate, which were heated to 60° C. 165.93 g of isophorone diisocyanate were then slowly added dropwise with stirring over 3 hours. Subsequently, stirring was continued until the NCO content had fallen below 0.2% (24 hours). A pale yellow resin with a residual NCO content of 0.13% and a viscosity of 6280 mPa·s (23° C.) was obtained.

1-10. (canceled)

11. Process for heterogeneously catalysed partial esterification of (meth)acrylic acid with oxyalkylated polyols, said oxyalkylated polyols having at least three free hydroxyl groups, characterized in that the catalyst is selected from the group comprising acidic ion exchange resins and/or acidic zeolites.

12. Process according to claim 11, wherein the oxyalkylated polyols have a degree of oxyalkylation of ≧1 to ≦30, preferably of ≧3 to ≦25.

13. Process according to claim 11, wherein the catalyst further comprises a stabilizer which is capable at least of slowing the polymerization of (meth)acrylic acid and wherein the concentration of the stabilizer on and/or in the catalyst is higher than the concentration of the stabilizer in the reaction solution.

14. Process according to claim 11, wherein the reaction conditions are selected from the group comprising:
the pressure is ≧0.5 bar to ≦5 bar, preferably ≧0.9 bar to ≦2 bar;
the temperature is ≧50° C. to ≦150° C., preferably ≧80° C. to ≦120° C.; and/or
the molar ratio of (meth)acrylic acid to OH groups is ≧1:3 to ≦3:3, preferably ≧1.5:3 to ≦2.5:3.

15. Process according to claim 11, wherein the catalyst is immobilized in a distillation column.

16. Process according to claim 11, wherein the catalyst is suspended in the reaction medium.

17. Process according to claim 11, wherein the catalyst is immobilized in the reaction medium.

18. Process according to claim 11, wherein the esterification is followed by the reaction of the resulting (meth)acrylic ester with an isocyanate, preferably with isophorone diisocyanate, hexamethylene 1,6-diisocyanate (HDI), diphenylmethane 4,4′-diisocyanate (MDI), tolylene 2,4-diisocyanate (TDI) and/or tolylene 2,6-diisocyanate (TDI).

19. (Meth)acrylic ester obtained by a process according to claim 11.

20. Use of (meth)acrylic esters according to claim 19 as radiation-curable compounds, preferably as binders for coating surfaces and/or articles.
